(19) United States
(12) Patent Application Publication
(10) Pub. No.: US 2024/0325542 A1
(43) Pub. Date: Oct. 3, 2024

(54) USE OF A PHOSPHOLIPID AS A CRYSTALLIZATION INHIBITOR

(71) Applicant: ZHEJIANG CUIZE PHARMACEUTICAL TECHNOLOGY CO., LTD., Hangzhou (CN)

(72) Inventors: Siping SUN, Hangzhou (CN); Jiamin LUO, Hangzhou (CN); Qiang GE, Hangzhou (CN); Cheng LIU, Hangzhou (CN)

(21) Appl. No.: 18/620,532

(22) Filed: Mar. 28, 2024

(30) Foreign Application Priority Data
Mar. 30, 2023 (CN) 202310356073.3

(51) Int. Cl. A61K 47/24; A61K 9/00; A61K 9/19; A61K 31/4174; A61K 31/4196; A61K 31/496; A61K 31/506

(52) U.S. Cl. CPC A61K 47/24; A61K 9/0075; A61K 9/19; A61K 31/4174; A61K 31/4196; A61K 31/496; A61K 31/506

(57) ABSTRACT

The present disclosure relates to the use of a phospholipid as a crystallization inhibitor. The present disclosure has discovered that a phospholipid has an effect of inhibiting crystallization, and by utilizing the crystallization-inhibiting effect of the phospholipid, the crystal growth of a pharmaceutical active ingredient is inhibited and the dispersion degree and delivery efficiency of the pharmaceutical active ingredient are improved, thereby making the pharmaceutical active ingredient druggable.

### CROSS-REFERENCE TO RELATED APPLICATIONS

This application claims the priority to Chinese Application No. CN202310356073.3, filed on Mar. 30, 2023. The content of the prior Chinese applications is considered as a part of the present disclosure and is incorporated herein in its entirety.

### TECHNICAL FIELD

The present disclosure relates to the field of pharmaceutical excipients, and in particular to the use of a phospholipid as a crystallization inhibitor.

### BACKGROUND

Pulmonary drug delivery has gradually drawn the attention from the medical community due to its advantages such as a large absorption area, a high drug availability, and small toxic side effects. However, pulmonary drug delivery is easily affected by various factors. In order to improve therapeutic effects, it is necessary to increase the lung deposition rate of drugs and enhance the drug stability by taking corresponding measures.

An inhalable pharmaceutical powder formulation (also known as dry powder inhalation (DPI)), is a special dosage form administered by pulmonary drug delivery, has become a hot topic in the research and development of formulations administered by pulmonary drug delivery in recent years, and has advantages such as easy use, no propellants and air pollution, low excipient dose, and high drug loadings. However, the existing pharmaceutical powder formulations have a physical property of poor dispersion between particles, and the use of traditional direct pulverization has the shortcomings such as a low yield, low delivery efficiency and poor stability, making it difficult to achieve desired therapeutic effects.

Phospholipids, a class of lipids containing phosphoric acid, are important amphiphilic substances, important components of biological membranes, and also are commonly used as emulsifiers and surfactants.

### SUMMARY

Given this, the present disclosure unexpectedly discovers the use of a phospholipid as a crystallization inhibitor by researching, which can inhibit the crystal growth of a pharmaceutical active ingredient, thereby improving the dispersion degree and delivery efficiency of the pharmaceutical active ingredient, and making the pharmaceutical active ingredient druggable.

According to an embodiment of the present disclosure, the use of a phospholipid as a crystallization inhibitor can be provided.

According to an embodiment of the present disclosure, the use of a phospholipid as a crystallization inhibitor in inhibition of the crystal growth of a pharmaceutical active ingredient can be provided.

### Beneficial Effects

The present disclosure provides the use of a phospholipid as a crystallization inhibitor. By utilizing the crystallization-inhibiting effect of the phospholipid, the particle size of crystal habit of the pharmaceutical active ingredient can be reduced, and the pulmonary delivery efficiency of the pharmaceutical active ingredient can be improved, which are conducive to preparing the pharmaceutical active ingredient into an inhalable pharmaceutical powder formulation, thereby achieving desired therapeutic effects.

### BRIEF DESCRIPTION OF THE DRAWINGS

To describe the technical solutions in the examples of the present disclosure more clearly, a brief introduction will be given below about the drawings required for examples description.

FIG. 1 shows the NGI measurement results of the powder formulation of example 1.

FIG. 2 shows the scanning electron microscope measurement results of the powder formulation of example 1.

FIG. 3 shows the NGI measurement results of the powder formulation of example 2.

FIG. 4 shows the scanning electron microscope measurement results of the powder formulation of example 2.

FIG. 5 shows the NGI measurement results of the powder formulation of example 3.

FIG. 6 shows the scanning electron microscope measurement results of the powder formulation of example 3.

FIG. 7 shows the NGI measurement results of the powder formulation of example 4.

FIG. 8 shows the scanning electron microscope measurement results of the powder formulation of example 4.

FIG. 9 shows the NGI measurement results of the powder formulation of example 5.

FIG. 10 shows the scanning electron microscope measurement results of the powder formulation of example 5.

FIG. 11 shows the NGI measurement results of the powder formulation of example 6.

FIG. 12 shows the NGI measurement results of the powder formulation of example 7.

FIG. 13 shows the NGI measurement results of the powder formulation of example 8.

FIG. 14 shows the NGI measurement results of the powder formulation of example 9.

FIG. 15 shows the NGI measurement results of the powder formulation of example 10.

FIG. 16 shows the NGI measurement results of the powder formulation of example 11.

FIG. 17 shows the NGI measurement results of the powder formulation of example 12.

FIG. 18 shows the scanning electron microscope measurement results of the powder formulation of comparative example 1.

FIG. 19 shows the NGI measurement results of the powder formulation of comparative example 1.

### DETAILED DESCRIPTION OF EMBODIMENTS

Unless otherwise indicated, all numbers representing content, concentration, ratio, weight, particle diameter, percentage, technical effect, and so forth as used in the description and claims are to be understood as being modified in any case by the term “about” or “approximately”. Accordingly, unless indicated to the contrary, numerical parameters set forth in the following specification and attached claims are approximations. Unless otherwise specified, the terms used herein have the meanings commonly understood by those skilled in the pertinent technical field. For those skilled in the art, each numerical parameter may vary depending upon the desired properties and effects sought to be obtained by the present disclosure and should be construed in light of the significant figures of digits and ordinary rounding techniques or in a manner understood by those skilled in the art.

Although the broad range of the numerical values and the parameters which are approximations of the present disclosure are as set forth herein, the numerical values as set forth in the specific examples are given as precisely as possible. However, any numerical value inherently contains certain errors, which are inevitably caused by the standard deviation found in their respective test measurements. Every numerical range given throughout the present description will include every narrower numerical range that falls within such a broader numerical range, as if such narrower numerical ranges are all expressly written herein.

As used herein, the expression “A and/or B” includes three cases: (1) A; (2) B; and (3) A and B. The expression “A, B and/or C” includes seven cases:
(1) A; (2) B; (3) C; (4) A and B; (5) A and C; (6) B and C; and (7) A, B and C. The meaning of similar expressions can be deduced in the same way.

As used herein, the term “aerodynamic particle size (aerodynamic diameter Da)”, also referred to as aerodynamic equivalent diameter, is a hypothetical particle size (particle diameter) that expresses the motion of the particles. It is defined by W. Stober as the diameter at which a sphere with a unit density (ρ₀=1 g/cm³) reaches the same final settling velocity (Vs) as an actual particle when performing motion at a low Reynolds number in still air. That is, the particle size of the actual particle is replaced with an equivalent diameter with the same aerodynamic characteristics. Generally, the particle size and density of the actual particle cannot be measured; however, the aerodynamic diameter can be measured directly by a dynamic method, so that the particle size of particles with different shapes, densities, optical and electrical properties can be measured in an unified manner. The aerodynamic diameter can be calculated with reference to the following method: the particle size (volume particle size) of a powder sample Dv is measured by a laser particle size analyzer, and the aerodynamic diameter Da is calculated according to Da=(ρ/ρ₁)^{1/2}X Dv, wherein ρ is the density of the particle, ρ₁=1 g/cm³, and Dv is the average particle size of the particle. The ρ value can be estimated from a tap density, and ρ is about 1.26 times the tap density.

As used herein, the term “mass median aerodynamic particle size” or “MMAD (mass median aerodynamic diameter)” refers to a particle size when the total mass of particles having various sizes smaller than a certain aerodynamic particle size in particulate matters accounts for 50% of the mass of all the particulate matters (i.e., the sum of the masses of all particles having different sizes).

As used herein, the term “fine particle fraction” or “FPF (fine particle fraction)” refers to the percentage of the dose of particles having a particle size less than or equal to 5 μm to the recovered dose, calculated as follows:

$$FPF = \frac{FPD}{Recovered dose} \times 100 \%$$

Wherein:

FPD refers to a fine particle dose, i.e., the dose of particles having a mass median aerodynamic particle size less than or equal to 5 μm, calculated according to the masses of drugs entering each stage of ACI or NGI and the corresponding cut-off particle size of each stage at a test flow rate;
the recovered dose refers to the sum of the masses of capsule residues, device residues, and drugs entering each stage of ACI or NGI.

A higher fine particle fraction indicates a higher pulmonary delivery efficiency.

As used herein, the term “crystallization inhibitor” refers to a compound that inhibits the growth of crystals, and refers to a compound for the present disclosure that inhibits the crystal growth of a pharmaceutical active ingredient.

As used herein, the term “crystal habit” has the meaning as commonly understood in the art and refers to a crystallization habit exhibited by a specific crystal variety on the crystal shape during spontaneous growth under normal external conditions. The stability of the corresponding formulation can be determined through the shape of the crystal habit observed by means of an electron microscope. For example, if the crystal habit is a blocky particle, it indicates that the corresponding formulation has an excellent stability.

As used herein, the term “small-molecule compound” refers to a compound with a molecular weight of less than 1,000 Da.

As used herein, the term “medium-molecule compound” refers to a compound with a molecular weight of greater than or equal to 1,000 Da and less than 5,000 Da.

As used herein, the term “macromolecular compound” refers to a compound with a molecular weight of greater than 5,000 Da.

The existing pharmaceutical powder formulations have a physical property of poor dispersion between particles, and the method by which a drug is directly pulverized has shortcomings such as a low yield, low delivery efficiency and poor stability, making it difficult to achieve desired therapeutic effects. The physicochemical properties, especially the morphology, of the pharmaceutical active ingredient are closely related to the physical properties and therapeutic effects of the pharmaceutical powder formulation. The present disclosure unexpectedly discovers the use of a phospholipid as a crystallization inhibitor. In the present disclosure, by utilizing the crystallization-inhibiting effect of the phospholipid, the particle size of crystal habit of the pharmaceutical active ingredient is reduced, and the pulmonary delivery efficiency of the pharmaceutical active ingredient is improved. The findings of the present disclosure are conducive to preparing the pharmaceutical active ingredient into an inhalable pharmaceutical powder formulation, thereby achieving desired therapeutic effects.

According to an embodiment of the present disclosure, the use of a phospholipid as a crystallization inhibitor can be provided.

According to an embodiment of the present disclosure, the use of a phospholipid as a crystallization inhibitor in inhibition of the crystal growth of an pharmaceutical active ingredient can be provided.

In some embodiments of the present disclosure, the pharmaceutical active ingredient is selected from a small-molecule compound, a medium-molecule compound or a macromolecular compound.

In some embodiments of the present disclosure, the pharmaceutical active ingredient is selected from the small-molecule compound or the medium-molecule compound.

In some embodiments of the present disclosure, the small-molecule compound is selected from an antifungal small-molecule compound, and the medium-molecule compound is selected from an antifungal medium-molecule compound.

In some embodiments of the present disclosure, the pharmaceutical active ingredient is selected from at least one of miconazole, fluconazole, voriconazole or itraconazole. Fungal infections are mostly caused by inhalation of fungal spores into airways, and the lung is a high-risk site for invasive fungal infections. In the present disclosure, by utilizing the inhibitory effect of a phospholipid on the crystal growth of the pharmaceutical active ingredient, the particle size of the crystal habit of the pharmaceutical active ingredient can be reduced, thereby reducing the bulk density and viscosity of the pharmaceutical active ingredient and improving the pulmonary delivery efficiency of the pharmaceutical active ingredient. Therefore, when the pharmaceutical active ingredient is the small-molecule compound or the medium-molecule compound resistant to fungal infections, it can ensure that the drug reaches the lung in a certain concentration to effectively exert the therapeutic efficacy thereof.

In some embodiments of the present disclosure, the pharmaceutical active ingredient is selected from voriconazole. Voriconazole is a triazole drug with characteristics of a broad antimicrobial spectrum and strong antimicrobial efficacy. Currently commercially available dosage forms are tablets and injections. Oral and injectable voriconazole is administered in large doses for a long medication cycle. However, voriconazole has a high nephrotoxicity and hepatotoxicity and severe side effects. Preparing pure voriconazole into a powder formulation by means of pulverizing has problems of low recovery and poor dispersibility, and thus voriconazole is not druggable. In the present disclosure, by utilizing the inhibitory effect of a phospholipid on the crystal growth of the pharmaceutical active ingredient, the particle size of the crystal habit of the pharmaceutical active ingredient can be reduced, thereby reducing the bulk density and viscosity of the pharmaceutical active ingredient and improving the pulmonary delivery efficiency of the pharmaceutical active ingredient. Therefore, compared with other types of pharmaceutical active ingredients, using voriconazole as the pharmaceutical active ingredient of the present disclosure is expected to reduce the administration dose while achieving a good fungal infection therapeutic effect, thereby greatly reducing the toxic side effects of the drug on patients.

In some embodiments of the present disclosure, the phospholipid is selected from at least one of phosphatidylcholine, phosphatidylethanolamine, phosphatidylserine, phosphatidylglycerol, phosphatidylinositol or cardiolipin.

In some embodiments of the present disclosure, the phospholipid is selected from at least one of phosphatidylcholine, phosphatidylethanolamine, phosphatidylglycerol or phosphatidylinositol.

As used herein, the term “phospholipid” refers to a lipid containing phosphoric acid, with a hydrophilic head composed of substituents linked to phosphoric acid and a hydrophobic tail composed of fatty acids. The phospholipid of the present disclosure has variable fatty acids, e.g., saturated fatty acids with 16-24 carbon atoms or unsaturated fatty acids with 16-24 carbon atoms, and variable substituents linked to phosphoric acid, e.g., choline, ethanolamine, serine, glycerol or inositol.

In some embodiments of the present disclosure, the phospholipid is selected from at least one of phosphatidylcholine or phosphatidylglycerol.

In some embodiments of the present disclosure, the phosphatidylcholine is selected from but not limited to at least one of egg yolk phosphatidylcholine, hydrogenated egg yolk phosphatidylcholine, soybean phosphatidylcholine, hydrogenated soybean lecithin, dilauroyl phosphatidylcholine, dimyristoyl phosphatidylcholine, dipalmitoyl phosphatidylcholine, distearoyl phosphatidylcholine, diolcoyl phosphatidylcholine, palmitoyl-oleoyl phosphatidylcholine, dilinoleoyl phosphatidylcholine, dilinolenoyl phosphatidylcholine, didecanoyl phosphatidylcholine, dierucoyl phosphatidylcholine, biseicosaoyl phosphatidylcholine, biseicosatrienoyl phosphatidylcholine, biseicosapentaenoyl phosphatidylcholine, bisdocosaenoyl phosphatidylcholine or bisdocosahexaenoyl phosphatidylcholine.

In some embodiments of the present disclosure, the phosphatidylethanolamine is selected from but not limited to at least one of egg yolk phosphatidylethanolamine, hydrogenated egg yolk phosphatidylethanolamine, soybean phosphatidylethanolamine, hydrogenated soybean phosphatidylethanolamine, dilauroyl phosphatidylethanolamine, dimyristoyl phosphatidylethanolamine, dipalmitoyl phosphatidylethanolamine, distearoyl phosphatidylethanolamine, dioleoyl phosphatidylethanolamine, palmitoyl-oleoyl phosphatidylethanolamine, dilinoleoyl phosphatidylethanolamine, dilinolenoyl phosphatidylethanolamine, didecanoyl phosphatidylethanolamine, dierucoyl phosphatidylethanolamine, biseicosaoyl phosphatidylethanolamine, biseicosatrienoyl phosphatidylethanolamine, biseicosapentaenoyl phosphatidylethanolamine, bisdocosaenoyl phosphatidylethanolamine or bisdocosahexaenoyl phosphatidylethanolamine.

In some embodiments of the present disclosure, the phosphatidylserine is selected from but not limited to at least one of soybean phosphatidylserine, hydrogenated soybean phosphatidylserine, dilauroyl phosphatidylserine, dimyristoyl phosphatidylserine, dipalmitoyl phosphatidylserine, distearoyl phosphatidylserine, diolcoyl phosphatidylserine, palmitoyl-oleoyl phosphatidylserine, dilinoleoyl phosphatidylserine, dilinolenoyl phosphatidylserine, didecanoyl phosphatidylserine, dierucoyl phosphatidylserine, biseicosaoyl phosphatidylserine, biseicosatrienoyl phosphatidylserine, biseicosapentaenoyl phosphatidylserine, bisdocosaenoyl phosphatidylserine or bisdocosahexaenoyl phosphatidylserine.

In some embodiments of the present disclosure, the phosphatidylglycerol is selected from but not limited to at least one of egg yolk phosphatidylglycerol, hydrogenated egg yolk phosphatidylglycerol, soybean phosphatidylglycerol, hydrogenated soybean phosphatidylglycerol, dilauroyl dimyristoyl phosphatidylglycerol, dipalmitoyl phosphatidylglycerol, distearoyl phosphatidylglycerol, dioleoyl phosphatidylglycerol, palmitoyl-oleoyl phosphatidylglycerol, dilinoleoyl phosphatidylglycerol, dilinolenoyl phosphatidylglycerol, didecanoyl phosphatidylglycerol, dierucoyl phosphatidylglycerol, biseicosaoyl phosphatidylglycerol, biseicosatrienoyl phosphatidylglycerol, biseicosapentaenoyl phosphatidylglycerol, bisdocosaenoyl phosphatidylglycerol or bisdocosahexaenoyl phosphatidylglycerol.

In some embodiments of the present disclosure, the phosphatidylinositol is selected from but not limited to at least one of phosphatidylinositol (CAS number: 97281-52-2), egg yolk phosphatidylinositol, hydrogenated egg yolk phosphatidylinositol, soybean phosphatidylinositol, hydrogenated soybean phosphatidylinositol, dilauroyl phosphatidylinositol, dimyristoyl phosphatidylinositol, dipalmitoyl phosphatidylinositol, distearoyl phosphatidylinositol, diolcoyl phosphatidylinositol, palmitoyl-oleoyl phosphatidylinositol, dilinolcoyl phosphatidylinositol, dilinolenoyl phosphatidylinositol, didecanoyl phosphatidylinositol, dierucoyl phosphatidylinositol, biseicosaoyl phosphatidylinositol, biseicosatrienoyl phosphatidylinositol, biseicosapentaenoyl phosphatidylinositol, bisdocosaenoyl phosphatidylinositol or bisdocosahexaenoyl phosphatidylinositol.

In some embodiments of the present disclosure, the cardiolipin is selected from but not limited to at least one of egg yolk cardiolipin, hydrogenated egg yolk cardiolipin, soybean cardiolipin, hydrogenated soybean cardiolipin, dilauroyl cardiolipin, dimyristoyl cardiolipin, dipalmitoyl cardiolipin, distearoyl cardiolipin, dioleoyl cardiolipin, palmitoyl-oleoyl cardiolipin, dilinoleoyl cardiolipin, dilinolenoyl cardiolipin, didecanoyl cardiolipin, dierucoyl cardiolipin, biseicosaoyl cardiolipin, biseicosatrienoyl cardiolipin, biseicosapentaenoyl cardiolipin, bisdocosaenoyl cardiolipin or bisdocosahexaenoyl cardiolipin.

In some embodiments of the present disclosure, the phospholipid is selected from at least one of dipalmitoyl phosphatidylcholine, hydrogenated soybean lecithin, distearoyl phosphatidylcholine, distearoyl phosphatidylglycerol, distearoyl phosphatidylethanolamine, phosphatidylinositol (CAS number: 97281-52-2) or egg yolk lecithin.

In some embodiments of the present disclosure, the phospholipid is selected from at least one of dipalmitoyl phosphatidylcholine, hydrogenated soybean lecithin, distearoyl phosphatidylcholine, distearoyl phosphatidylglycerol, distearoyl phosphatidylethanolamine or egg yolk lecithin.

In some embodiments of the present disclosure, the phospholipid is selected from at least one of hydrogenated soybean lecithin, distearoyl phosphatidylcholine, distearoyl phosphatidylglycerol, distearoyl phosphatidylethanolamine or egg yolk lecithin.

In some embodiments of the present disclosure, the phospholipid is selected from at least one of hydrogenated soybean lecithin, distearoyl phosphatidylcholine or distearoyl phosphatidylglycerol.

In some embodiments of the present disclosure, the mass ratio of the pharmaceutical active ingredient to the crystallization inhibitor is in a range of 1:1 to 100:1.

In some embodiments of the present disclosure, the mass ratio of the pharmaceutical active ingredient to the crystallization inhibitor is in a range of 2:1 to 100:1. In some embodiments of the present disclosure, the mass ratio of the pharmaceutical active ingredient to the crystallization inhibitor is in a range of 5:1 to 100:1. In some embodiments of the present disclosure, the mass ratio of the pharmaceutical active ingredient to the crystallization inhibitor is in a range of 9:1 to 100:1. Specifically, the mass ratio of the pharmaceutical active ingredient to the crystallization inhibitor is 99:1, 80:1, 70:1, 60:1, 50:1, 40:1, 30:1, 20:1, 15:1 or 10:1.

In some embodiments of the present disclosure, the pharmaceutical active ingredient, the crystallization inhibitor and/or other pharmaceutically acceptable excipients are dissolved in a solvent to obtain a mixed solution, and the mixed solution is lyophilized to obtain a lyophilized powder.

In some embodiments of the present disclosure, the lyophilized powder is pulverized.

In some embodiments of the present disclosure, the other pharmaceutically acceptable excipients include a stabilizer.

In some embodiments of the present disclosure, the stabilizer includes a salt, e.g., at least one selected from an inorganic salt or an organic salt. In some embodiments of the present disclosure, the inorganic salt includes at least one of sodium chloride, potassium chloride, calcium chloride, magnesium chloride, barium chloride, sodium sulfate, magnesium sulfate, calcium sulfate, calcium sulfate, potassium sulfate, sodium phosphate or potassium phosphate. In some embodiments of the present disclosure, the stabilizer includes the inorganic salt that can provide a monovalent and divalent metal cation, e.g., at least one of sodium chloride, calcium chloride, sodium sulfate and calcium sulfate, or includes at least one of calcium chloride and sodium chloride.

In some embodiments of the present disclosure, the solvent comprises water and an organic solvent.

In some embodiments of the present disclosure, the organic solvent includes alcohol compound. In some embodiments of the present disclosure, the alcohol compound includes tert-butyl alcohol.

In some embodiments of the present disclosure, the solvent includes an aqueous tert-butyl alcohol solution.

In some embodiments of the present disclosure, in the aqueous tert-butyl alcohol solution, the mass ratio of tert-butyl alcohol to water is 1:10-10:1. In some embodiments of the present disclosure, in the aqueous tert-butyl alcohol solution, the mass ratio of tert-butyl alcohol to water is 7:3.

In some embodiments of the present disclosure, in the aqueous tert-butyl alcohol solution, the mass ratio of tert-butyl alcohol to water c be 1:9, 1:8, 1:7, 1:6, 1:5, 1:4, 1:3, 1:2, 1:1, 2:1, 3:1, 4:1, 5:1, 6:1, 7:1, 8:1, 9:1 or a value in a range composed of any two points above.

In some embodiments of the present disclosure, under the same conditions, the lyophilized powder has a reduced particle size of crystal habit compared to that prepared with the solution without the crystallization inhibitor.

In some embodiments of the present disclosure, the lyophilized powder has a mass median aerodynamic particle size of 0.5 μm-10 μm.

In some embodiments of the present disclosure, the lyophilized powder has a mass median aerodynamic particle size of 0.5 μm-5 μm.

In some embodiments of the present disclosure, the lyophilized powder has a mass median aerodynamic particle size of 0.5 μm-6 μm, 0.5 μm-4.8 μm, 0.5 μm-4.5 μm, 0.5 μm-4μ, 0.5 μm-3.5 μm, 0.5 μm-3 μm, 0.5 μm-2.5 μm or 0.5 μm-2 μm. In some embodiments of the present disclosure, the lyophilized powder has a mass median aerodynamic particle size of 5 μm, 4.7 μm, 4.4 μm, 4.2 μm, 3.8 μm, 3.6 μm, 3.4 μm, 3.2 μm or 2.5 μm.

In some embodiments of the present disclosure, the crystallization inhibitor is used to prepare an inhalable pharmaceutical powder formulation.

In some embodiments of the present disclosure, the content of the pharmaceutical active ingredient per unit dose of the pharmaceutical powder formulation is 5 mg-100 mg, e.g., 5 mg-50 mg, 5 mg-30 mg, or 5 mg-20 mg. Within the unit dose range, it will facilitate the production and exertion of therapeutic effects of drugs. It can be understood that the content of the pharmaceutical active ingredient per unit dose of the pharmaceutical powder formulation could be any value in a range of 5 mg-100 mg, e.g., 5 mg, 10 mg, 15 mg, 20 mg, 30 mg, 40 mg, 50 mg, 60 mg, 70 mg, 80 mg, 90 mg, 100 mg, etc., or other unlisted values in the range of 5 mg-100 mg.

In some embodiments of the present disclosure, the pharmaceutical powder formulation can be filled into a capsule and can be inhaled into the lung by means of the capsule, or can also be directly inhaled into the lung by means of an inhalation device.

The above-mentioned various embodiments and preferences for the pharmaceutical powder formulation of the present disclosure can be combined with each other (as long as they are not inherently contradictory to each other), and are also applicable to the preparation method for the pharmaceutical powder formulation of the present disclosure, and the various embodiments formed by the combination are considered as a part of the present disclosure.

The technical solution of the present disclosure will be more clearly and explicitly described by way of illustration in combination with examples. It should be understood that these examples are only for illustrative purposes and not intended to limit the protection scope of the present disclosure. The protection scope of the present disclosure is only defined by the claims.

### EXAMPLES

### Materials and Methods

Voriconazole used in the examples was purchased from Sichuan RenAn Pharmaceutical Co., Ltd.; fluconazole was purchased from Jiangxi Revere Biotechnology Co., Ltd.; tert-butyl alcohol was purchased from Aladdin Reagent (Shanghai) Co., Ltd.; hydrogenated soybean lecithin was purchased from Nippon Fine Chemical Co., Ltd., Japan; dipalmitoyl phosphatidylcholine, distearoyl phosphatidylcholine and distearoyl phosphatidylglycerol were purchased from Jiangsu Southeast Nanomaterials Co., Ltd.; and egg yolk lecithin, distearoyl phosphatidylethanolamine and phosphatidylinositol (CAS number: 97281-52-2) were purchased from AVT (Shanghai) Pharmaceutical Tech. Co., Ltd.

The mass median aerodynamic particle size and the fine particle fraction were measured using an Andersen 8-stage impactor (ACI cascade sampler) or a next-generation 8-stage impactor (NGI cascade sampler), and the specific operational procedure was as follows: the lyophilized powder was filled into a No. 3 capsule, and a Breezhaler® inhaler device and a device adapter were used and connected to an artificial throat of an impactor and an air inlet end of a pre-separator; the air flow rate of a pump was adjusted to 60 L/min and the pumping time is set to 4 seconds; the capsule was punctured, and inhalation was started, so that the pharmaceutical powder entered different stages of the impactor along with the airflow; and the pharmaceutical powders in different stages of the impactor were washed with an aqueous methanol solution (the volume ratio of methanol to water was 7:3) into a volumetric flask and were subjected to determining capacity, and the samples were taken to detect the content of the pharmaceutical powder in each stage of the impactor by means of high-performance liquid chromatography.

The parameters for lyophilization in a lyophilizer were as follows:

| freezing control | Set temperature | Heating-up time | Duration |
|---|---|---|---|
| Phase 1 | −45° C. | 90 min | 300 min |

| | Set | | Heating-up | | | Set |
|---|---|---|---|---|---|---|
| | temperature | | time | Duration | | vacuum |
| Primary drying | | | | | | |
| Phase 1 | −10° | C. | 120 min | 1200 | min | 0.02 mbar |
| Desorption drying | | | | | | |
| Phase 1 | 20° | C. | 240 min | 1,500 | min | 0.02 mbar |

The parameters for pulverization in a disintegrator were as follows:

| Feed speed | Venturi pressure | Confining pressure |
|---|---|---|
| 1.0-2.5 | 3.0-6.0 bar | 3.5-6.5 bar |

### Examples 1-7: Preparation of Lyophilized Powders with Different Types of Phospholipids

a. Formulae, as Shown in the Table Below

| | The | Content of the | | |
|---|---|---|---|---|
| | pharmaceutical | pharmaceutical | The type of | Content of |
| No. | active ingredient | active ingredient | Phospholipid | phospholipid |
| Example | Voriconazole | 94.5% | Hydrogenated soybean | 5.5% |
| 1 | | | lecithin | |
| Example | Voriconazole | 94.5% | Dipalmitoyl | 5.5% |
| 2 | | | phosphatidylcholine | |
| Example | Voriconazole | 95.0% | Distearoyl | 5.0% |
| 3 | | | phosphatidylcholine | |
| Example | Voriconazole | 92.0% | Distearoyl | 8.0% |
| 4 | | | phosphatidylglycerol | |
| Example | Voriconazole | 92.0% | Egg yolk lecithin | 8.0% |
| 5 | | | | |
| Example | Voriconazole | 92.0% | Distearoyl | 8.0% |
| 6 | | | phosphatidylethanolamine | |
| Example | Voriconazole | 92.0% | Phosphatidylinositol | 8.0% |
| 7 | | | | |

b. Process

The pharmaceutical active ingredient and the phospholipid were dissolved in the aqueous tert-butyl alcohol solution (tert-butyl alcohol:water=7:3, mass ratio), lyophilized in a lyophilizer (LYO-0.4, Shanghai Tofflon Science and Technology Co., Ltd.), then taken out, and sieved through a 0.5 cm sieve. Pulverize in a jet mill (Mc Jet100, DEC Corporation).

c. Results

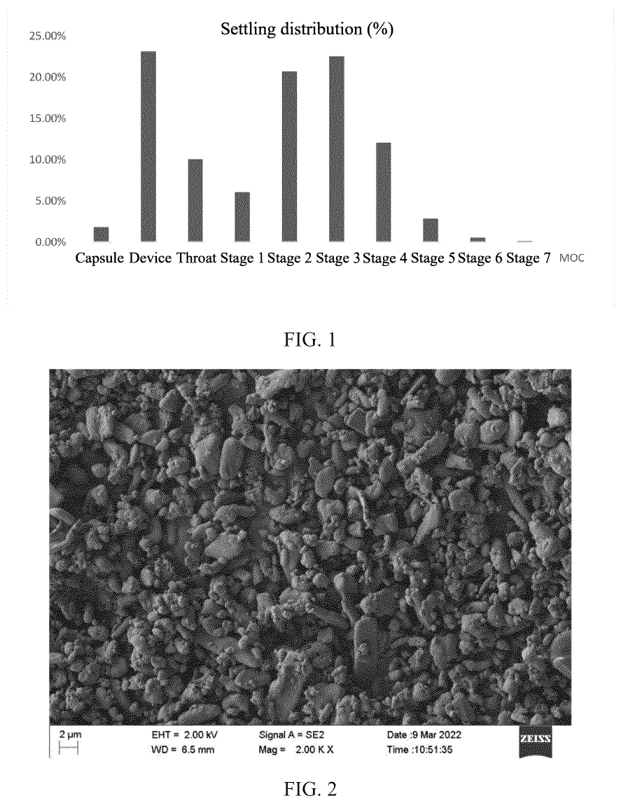

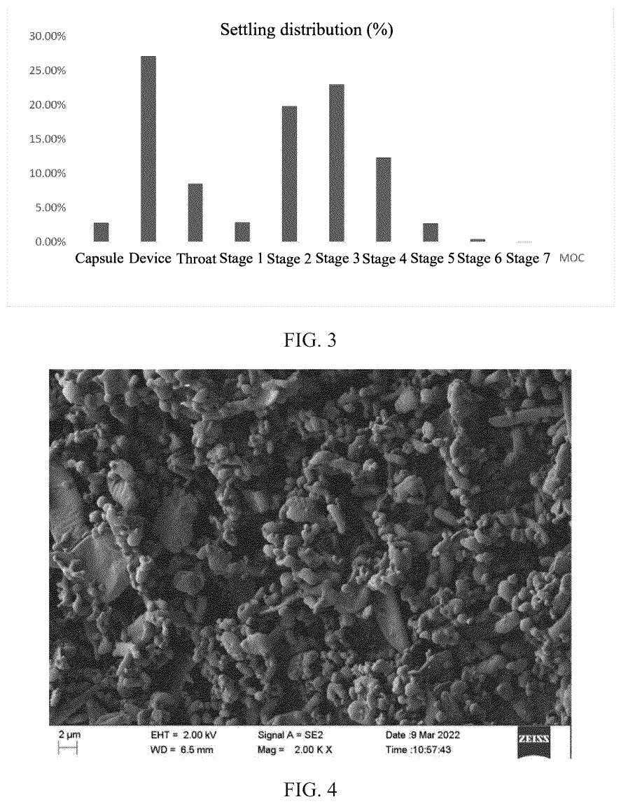

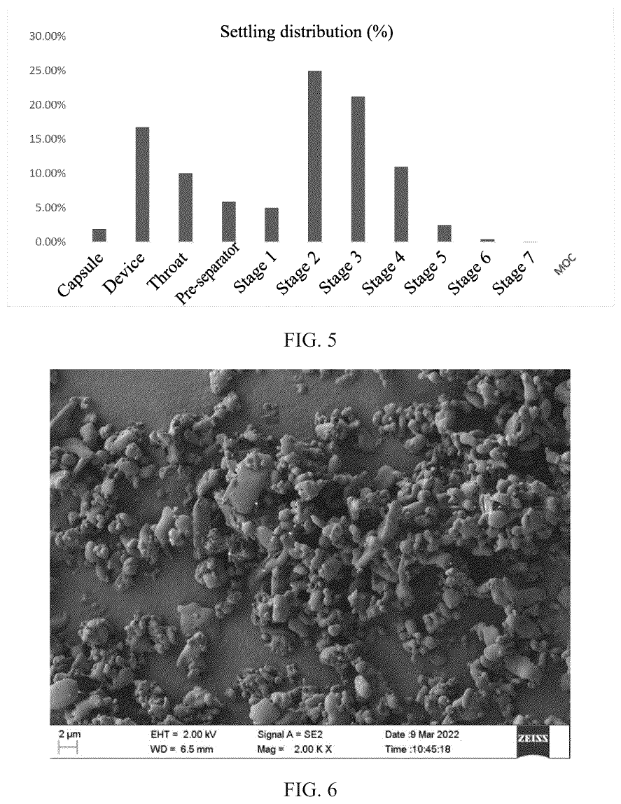

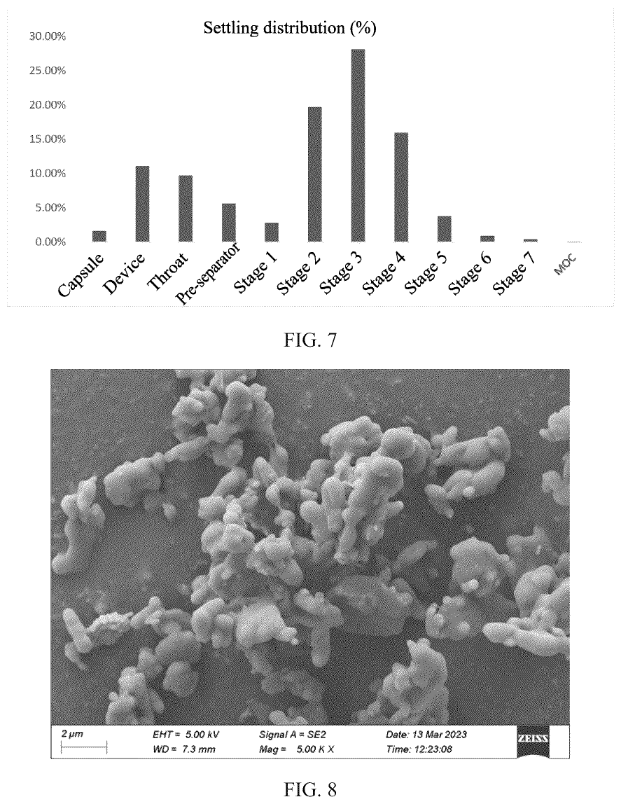

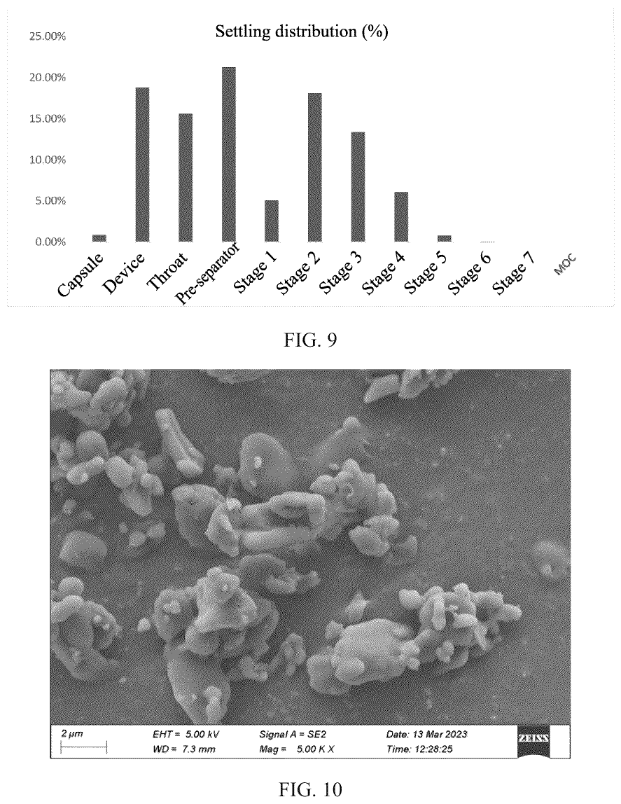

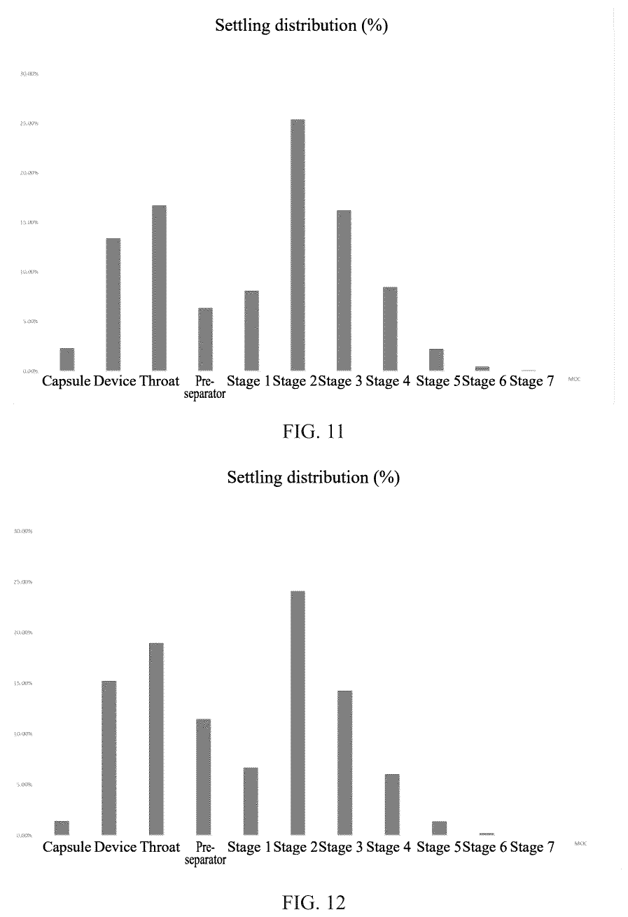

The NGI settling distribution results and the electron microscope scanning images of examples 1-5 were shown in FIGS. 1-10, and the NGI settling distribution results of examples 6-7 were shown in FIGS. 11-12. Upon calculation, the data of the fine particle fraction and the mass median aerodynamic particle size of examples 1-7 were as shown in the table below.

| | | | | Morphology under |
|---|---|---|---|---|
| | The type of | | MMAD | scanning electron |
| No. | Phospholipid | FPF | (μm) | microscope |
| Example 1 | Hydrogenated soybean | 43.41% | 4.040 | Spherical particles |
| | lecithin | | | |
| Example 2 | Dipalmitoyl | 44.38% | 3.827 | Spherical particles |
| | phosphatidylcholine | | | |
| Example 3 | Distearoyl | 41.56% | 4.242 | Spherical particles |
| | phosphatidylcholine | | | |
| Example 4 | Distearoyl | 55.20% | 3.589 | Spherical particles |
| | phosphatidylglycerol | | | |
| Example 5 | Egg yolk lecithin | 24.54% | 4.649 | Spherical particles |
| Example 6 | Distearoyl | 33.36% | 4.730 | / |
| | phosphatidylethanolamine | | | |
| Example 7 | Phosphatidylinositol | 27.33% | 4.899 | / |

### Examples 8-11: Preparation of Lyophilized Powders with Different Phospholipid Contents

a. Formulae, as Shown in the Table Below

| | The | Mass of the | | |
|---|---|---|---|---|
| | pharmaceutical | pharmaceutical | The type of | Mass of |
| No. | active ingredient | active ingredient | Phospholipid | phospholipid |
| Example 8 | Voriconazole | 99% | Distearoyl | 1% |
| | | | phosphatidylcholine | |
| Example 9 | Voriconazole | 98% | Distearoyl | 2% |
| | | | phosphatidylcholine | |
| Example 10 | Voriconazole | 95% | Distearoyl | 5% |
| | | | phosphatidylcholine | |
| Example 11 | Voriconazole | 92% | Distearoyl | 8% |
| | | | phosphatidylcholine | |

b. Process

The process was the same as that of example 1, with the only exception of different specific formulae.

c. Results

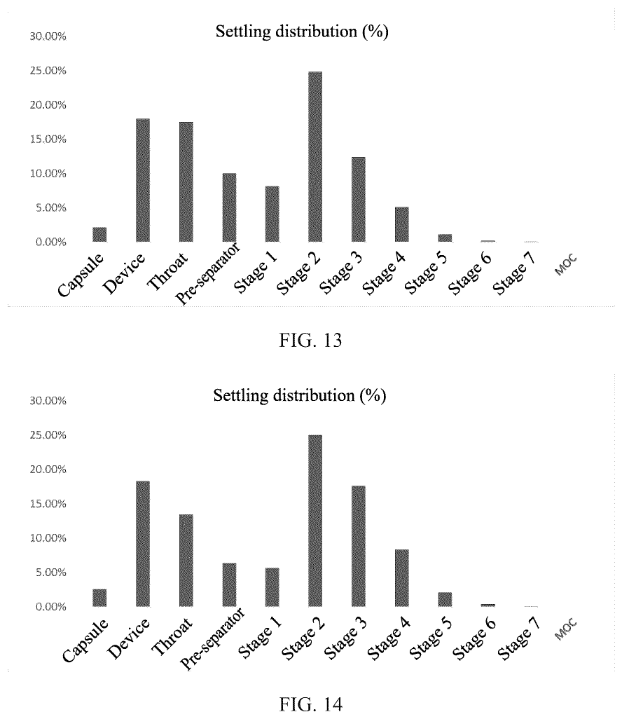

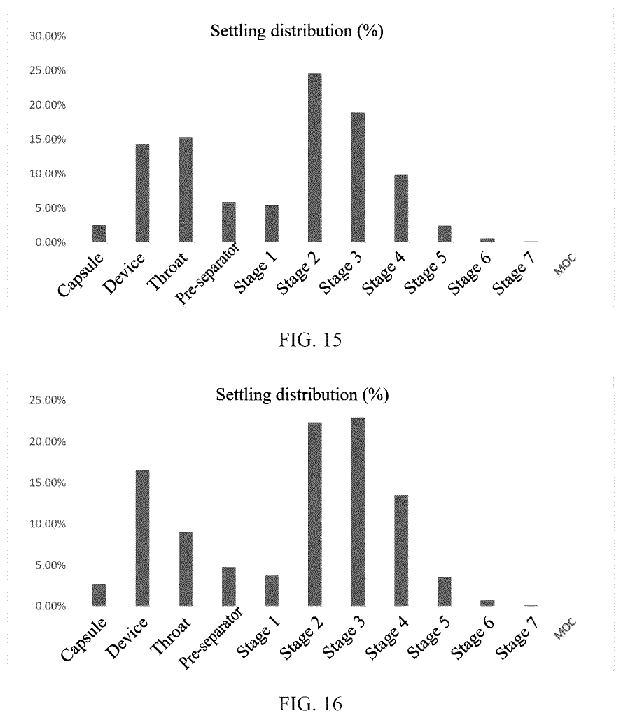

The NGI settling distribution results of examples 8-11 were shown in FIGS. 13-16. Upon calculation, the data of the fine particle fraction and mass median aerodynamic particle size of examples 8-11 were as shown in the table below.

| | The type of | Mass of | | MMAD |
|---|---|---|---|---|
| No. | Phospholipid | phospholipid | FPF | (μm) |
| Example 8 | Distearoyl | 1% | 24.39% | 5.180 |
| | phosphatidylcholine | | | |
| Example 9 | Distearoyl | 2% | 34.83% | 4.547 |
| | phosphatidylcholine | | | |
| Example 10 | Distearoyl | 5% | 38.21% | 4.368 |
| | phosphatidylcholine | | | |
| Example 11 | Distearoyl | 8% | 47.07% | 3.859 |
| | phosphatidylcholine | | | |

### Example 12: Preparation of Lyophilized Powders with Different Pharmaceutical Active Ingredients

a. Formula, as Shown in the Table Below

| | The | Mass of the | | |
|---|---|---|---|---|
| | pharmaceutical | pharmaceutical | The type of | Mass of |
| No. | active ingredient | active ingredient | Phospholipid | phospholipid |
| Example 12 | Fluconazole | 92.0% | Distearoyl | 8.0% |
| | | | phosphatidylcholine | |

b. Process

The process was the same as that of example 1, with the only exception of different specific formula.

c. Results

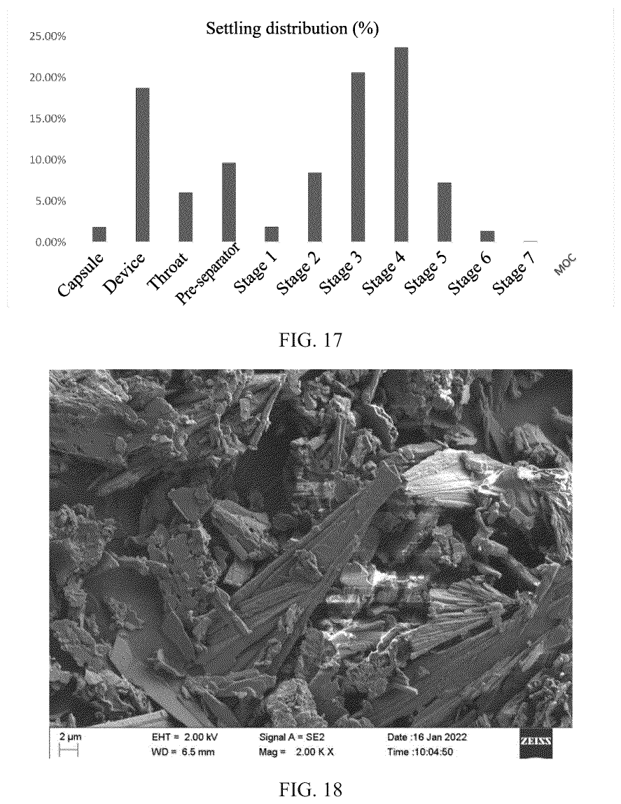

The NGI settling distribution results of example 12 were shown in FIG. 17. Upon calculation, the fine particle fraction and the mass median aerodynamic particle size of example 12 were as shown in the table below.

| | the pharmaceutical | | MMAD |
|---|---|---|---|
| No. | active ingredient | FPF | (μm) |
| Example 12 | Fluconazole | 61.88% | 2.617 |

### Comparative Example 1

### Process

24 g voriconazole was dissolved in an aqueous tert-butyl alcohol solution (tert-butyl alcohol:water=7:3, mass ratio), lyophilized in a lyophilizer (LYO-0.4, Shanghai Tofflon Science and Technology Co., Ltd.), then taken out, and sieved through a 0.5 cm sieve. Pulverize in a jet mill (Mc Jet100, DEC Corporation).

### Results

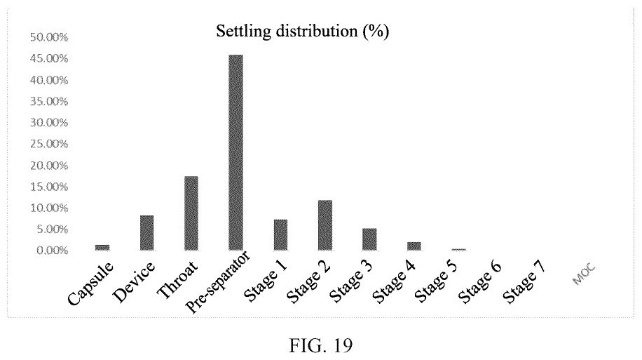

The NGI settling distribution results and the electron microscope scanning image of comparative example 1 were shown in FIGS. 18-19. Upon calculation, the fine particle fraction and the mass median aerodynamic particle size of comparative example 1 were as shown in the table below.

| | | MMAD | Morphology under scanning |
|---|---|---|---|
| No. | FPF | (μm) | electron microscope |
| Comparative example 1 | 9.87% | 6.128 | Flaky and needle-shaped |

According to the scanning electron microscope images of examples 1-5 and comparative example 1, it can be seen that the lyophilized powders of examples 1-5 were spheroidal particles with a smaller size, and the lyophilized powder of comparative example 1 were flaky and needle-shaped particles with a larger size, which illustratively proves that phospholipids have a better effect of inhibiting the crystal growth. Further, the lyophilized powders of examples 1-12 have a higher fine particle fraction and a smaller mass median aerodynamic particle size, which indicates that the crystal growth of the lyophilized powders of the present application is inhibited by phospholipids, thus improving the pulmonary delivery efficiency of the pharmaceutical active ingredient. In summary, it can be proven that phospholipids have a better effect of inhibiting the crystal growth and can be used as a crystallization inhibitor. The findings of the present disclosure are conducive to preparing the pharmaceutical active ingredient into an inhalable pharmaceutical powder formulation, thereby achieving desired therapeutic effects.

Although the specific embodiments have been described, for the applicant or a person skilled in the art, the substitutions, modifications, changes, improvements, and substantial equivalents of the above embodiments may exist or cannot be foreseen currently. Therefore, the submitted appended claims and claims that may be modified are intended to cover all such substitutions, modifications, changes, improvements, and substantial equivalents.

1. A method of inhibiting the crystal growth of a compound, comprising contacting the compound with a crystallization inhibitor comprising a phospholipid.

2. A method of inhibiting the crystal growth of a pharmaceutical active ingredient, comprising contacting the pharmaceutical active ingredient with a crystallization inhibitor comprising a phospholipid.

3. The method according to claim 1 or 2, wherein the phospholipid is selected from at least one of phosphatidylcholine, phosphatidylethanolamine, phosphatidylserine, phosphatidylglycerol, phosphatidylinositol or cardiolipin.

4. The method according to claim 1 or 2, wherein the phospholipid is selected from at least one of dipalmitoyl phosphatidylcholine, hydrogenated soybean lecithin, distearoyl phosphatidylcholine, distearoyl phosphatidylglycerol, distearoyl phosphatidylethanolamine or egg yolk lecithin.

5. The method according to claim 1 or 2, wherein the phospholipid is selected from at least one of hydrogenated soybean lecithin, distearoyl phosphatidylcholine, distearoyl phosphatidylglycerol, distearoyl phosphatidylethanolamine or egg yolk lecithin.

6. The method according to claim 2, wherein the pharmaceutical active ingredient is selected from a small-molecule compound or a medium-molecule compound.

7. The method according to claim 6, wherein the small-molecule compound is selected from an antifungal small-molecule compound, and the medium-molecule compound is selected from an antifungal medium-molecule compound.

8. The method according to claim 6, wherein the pharmaceutical active ingredient is selected from at least one of miconazole, fluconazole, voriconazole or itraconazole.

9. The method according to claim 6, wherein the mass ratio of the pharmaceutical active ingredient to the crystallization inhibitor is in a range of 1:1 to 100:1.

10. The method according to claim 2, wherein the pharmaceutical active ingredient, the crystallization inhibitor and/or other pharmaceutically acceptable excipients are dissolved in a solvent to obtain a mixed solution, and the mixed solution is lyophilized to obtain a lyophilized powder.

11. The method according to claim 10, wherein the solvent comprises water and an organic solvent.

12. The method according to claim 11, wherein under the same conditions, the lyophilized powder has a reduced particle size of crystal habit compared to that prepared with the solution without the crystallization inhibitor.

13. The method according to claim 12, wherein the lyophilized powder has a mass median aerodynamic particle size of 0.5 μm-10 μm.

14. The method according to claim 12, wherein the lyophilized powder has a mass median aerodynamic particle size of 0.5 μm-5 μm.

15. The method according to claim 1 or 2, wherein the crystallization inhibitor is used to prepare an inhalable pharmaceutical powder formulation.
